(19) United States
(12) Patent Application Publication
(10) Pub. No.: US 2009/0297626 A1
(43) Pub. Date: Dec. 3, 2009

(54) Methods for preparing metal oxides

(75) Inventors: Stephen O'Brien, New York, NY (US); Limin Huang, New York, NY (US)

Correspondence Address: SCHWEGMAN, LUNDBERG & WOESSNER, P.A., P.O. BOX 2938, MINNEAPOLIS, MN, 55402, US

(73) Assignee: The Trustees of Columbia University in the City of New York, New York, NY (US)

(21) Appl. No.: 11/982,842

(22) Filed: Nov. 5, 2007

Related U.S. Application Data
(60) Provisional application No. 60/856,707, filed on Nov. 3, 2006.

(51) Int. Cl. A61K 33/30; C01G 9/02; C01G 23/047; A61K 33/24; C09K 3/00; C07F 7/28; C07F 3/06

(52) U.S. Cl. 424/642; 423/622; 423/610; 424/641; 424/617; 252/182.33; 556/54; 556/130; 977/773

(57) ABSTRACT

The disclosed subject matter provides a method for preparing a metal oxide, the method includes (a) contacting a metal salt precursor with an alcohol to provide a metal oxide; and (b) removing the metal oxide from the alcohol.

### CROSS-REFERENCE TO RELATED APPLICATIONS

This application claims priority benefit to U.S. Provisional Application Nos. 60/856,707, filed Nov. 3, 2006; the entirety of which is incorporated herein by reference.

### STATEMENT REGARDING FEDERALLY SPONSORED RESEARCH FOR DEVELOPMENT

The present invention was made with United States government support under Grant No. CHE-01-17752 and DE-FG02-03ER15463 awarded by the National Science Foundation. The United States government may have certain rights in this invention.

### BACKGROUND

Metal oxide nanoparticles, such as ZnO and TiO₂ nanoparticles, have attracted much interest due to their unique optical, electrical and magnetic properties associated with quantum size effects. For example, ZnO and TiO₂ nanoparticles have gained much interest due to their ultraviolet (UV) light absorption properties.

Ultraviolet (UV) light from the sun is composed of UVA (320-400 nm) and UVB (290-320 nm). UVB, which is directly absorbed by the cell, has long been linked to sunburn, aging, and skin cancer. UVA has also recently been suspected of being involved in similar skin problems.

The traditional SPF (Sun Protection Factor) describes the performance of the products primarily in terms of UVB protection. A Star Rating System, which provides a measure of UVA protection in the form of UVA to UVB protection ratio, allows the consumer to gain a better picture of the performance of UV protection offered by the various products, such as cosmetic and sun care formulations.

Cosmetic formulations designed to absorb UV radiation are often formulated using a mixture of organic (e.g., dibenzoylmethanes and methoxycinnamates) or inorganic (e.g., TiO₂ or ZnO) UV absorbers. Generally, organic UV absorbers can show reduced long-term stability to UV light due to various chemical reactions being induced by either UV light or free radicals excited by sunlight. Inorganic UV absorbers, on the other hand, are not susceptible to degradation by sunlight. However, the inorganic UV absorbers can also form free radicals that can go on to attack the organics.

To overcome such problems, low levels of foreign elements were introduced into the inorganic UV absorbers. The dopants in the lattice were able to modify the bandgap of the inorganic system and were also able to trap any charges excited by UV light absorption within the inorganic particles. (See, e.g., Wakefield et al., “Modified titania nanomaterials for sunscreen applications—reducing free radical generation and DNA damage,” Materials Science and Technology, (2004), vol. 20, pp 985-988).

Due to the properties and advantages described above, various techniques to produce metal oxide nanoparticles have been reported (see, e.g., Niederberger et al., “Benzyl alcohol and titanium tetrachloride—a versatile reaction system for the nonaqueous and low-temperature preparation of crystalline and luminescent titania nanoparticles,” Chem. Mater., (2002), vol. 14, pp. 4364-4370; Viswanatha et al., “Synthesis and characterization of Mn-doped ZnO nanocrystal,” J. Phys. Chem. B., (2004), vol. 108, pp. 6303-6310; Zhang et al., “Synthesis of flower-like ZnO nanostructures by an organic-free hydrothermal process,” Nanotechnology, (2004), vol. 15, pp. 622-626; Spanhel et al., “Colloidal ZnO nanostructures and functional coatings: A survey,” J. of Sol-Gel Science and Technology, (2006), Vol. 39, pp. 7-24; and Yin et al., “Zinc Oxide Quantum Rods,” J. Am. Chem. Soc., (2004), Vol. 126, pp 6206-6207.

However, many of these currently existing techniques are inadequate. For example, certain synthetic techniques introduce foreign cationic species (e.g., Li⁺ or Na⁺ or K⁺) or anionic species (e.g. Cl⁻, Br⁻) that can change the electrical and luminescent properties of metal oxide nanoparticles. Moreover, the toxic or hazardous nature of organic solvents and ligand impurities that are utilized in certain synthetic techniques are an additional source of concern. In other synthetic techniques, reactions can proceed extremely fast, which can be dangerous, lead to less uniform size of nanoparticles, and lead to aggregated nanoparticles.

### BRIEF DESCRIPTION OF THE DRAWINGS

Embodiments of the disclosed subject matter may be best understood by referring to the following description and accompanying drawings which illustrate such embodiments. The numbering scheme for the Figures included herein are such that the leading number for a given reference number in a Figure is associated with the number of the Figure. For example, a chart diagram depicting the metal oxide (19) may be located in FIG. 10. In the drawings:

FIG. 1 illustrates an x-ray diffraction (XRD) pattern of ZnO nanoparticles.

FIG. 2 illustrates a transmission electron microscope (TEM) image of ZnO nanoparticles coated with oleic acid.

FIG. 3 illustrates a transmission electron microscope (TEM) image of Mn-doped ZnO nanoparticles.

FIG. 4 illustrates XRD patterns of ZnO nanoparticles and Mn-doped (3 mol %) ZnO nanoparticles.

FIG. 5 illustrates photos of ZnO nanoparticles and Mn-doped (3 mol %) ZnO nanoparticles dispersed in water.

FIG. 6 illustrates room temperature UV-vis absorption spectra of ZnO nanoparticles crystallized for five hours, ZnO nanoparticles crystallized for ten hours, and Mn-doped (3 mol %) ZnO nanoparticles crystallized for 10 hours.

FIG. 7 illustrates XRD patterns of TiO₂ nanoparticles synthesized in ethanol, Mn-doped (3 mol %) TiO₂ nanoparticles synthesized in ethanol, and TiO₂ nanocrystals synthesized in oleyl alcohol.

FIG. 8 illustrates a TEM image of TiO₂ nanoparticles synthesized in ethanol.

FIG. 9 illustrates a TEM image of TiO₂ nanoparticles synthesized in oleyl alcohol.

FIG. 10 illustrates a chart diagram that includes methods of making metal oxides.

### SUMMARY

The disclosed subject matter provides metal oxides, as well as methods of making and using the same. The method produces a relatively narrow size distribution of the metal oxide, e.g., in the nanometer range of about 5-20 nm. This size regime is difficult to achieve with conventional techniques, such as powder processing (e.g., grinding, milling, spray pyrolysis) or hydrothermal or sol gel processing. The methods of the presently disclosed subject matter are also relatively inexpensive and simple. Additionally, the methods of the presently disclosed subject matter typically include a one pot synthesis. The metal oxides obtained via the methods of the presently disclosed subject matter are highly dispersed in aqueous or alcoholic media, which are suitable for the electronics, pharmaceutical and cosmetic industries. Furthermore, the surface of the metal oxides obtained via the methods of the presently disclosed subject matter are compatible upon mixing with pharmaceutical and cosmetic carriers and diluents (e.g., phospholipids, PEG, liposomes, etc.).

The disclosed subject matter provides a method for preparing a metal oxide, the method includes (a) contacting a metal salt precursor with an alcohol to provide a metal oxide; and (b) removing the metal oxide from the alcohol.

The disclosed subject matter provides a method for preparing a metal oxide nanoparticle, the method includes (a) contacting a metal salt precursor with an alcohol to provide a metal oxide; (b) removing the metal oxide from the alcohol; (c) redispersing the metal oxide in a solvent to provide a colloidal suspension of the metal oxide and the solvent; and (d) removing the metal oxide from the solvent to provide a metal oxide nanoparticle including at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.

The disclosed subject matter provides a method for preparing a metal oxide nanoparticle, the method includes (a) contacting two or more metal salt precursors with an alcohol to provide a metal oxide that precipitates from the alcohol, wherein the metal salt precursor includes at least one of titanium acetylacetonate, titanium isopropoxide, zinc acetate, zinc citrate, zinc methacrylate, zinc oxalate, manganese acetate, cobalt acetate, and manganese acetylacetonate; (b) removing the precipitated metal oxide from the alcohol; (c) redispersing the precipitated metal oxide in a solvent to provide a colloidal suspension of the redispersed metal oxide and the solvent; and (d) removing the redispersed metal oxide from the solvent to provide a metal oxide nanoparticle including at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.

The disclosed subject matter provides a method for preparing a metal oxide nanoparticle, the method includes (a) contacting a metal salt precursor with an alcohol to provide a metal oxide that precipitates from the alcohol, wherein the metal salt precursor includes at least one of titanium acetylacetonate, titanium isopropoxide, zinc acetate, zinc citrate, zinc methacrylate, zinc oxalate, manganese acetate, cobalt acetate, and manganese acetylacetonate; (b) removing the precipitated metal oxide from the alcohol; (c) redispersing the precipitated metal oxide in a solvent to provide a colloidal suspension of the redispersed metal oxide and the solvent; and (d) removing the redispersed metal oxide from the solvent to provide a metal oxide nanoparticle including at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.

### DETAILED DESCRIPTION

The disclosed subject matter provides metal oxides, as well as methods of making and using the same.

Reference will now be made in detail to certain claims of the disclosed subject matter, examples of which are illustrated below. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which may be included within the scope of the disclosed subject matter as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The disclosed subject matter relates to metal oxides, as well as methods of making and using the same. When describing the metal oxides, as well as methods of making and using the same, the following terms have the following meanings, unless otherwise indicated.

### DEFINITIONS

Unless stated otherwise, the following terms and phrases as used herein are intended to have the following meanings:

As used herein, “metal oxide” refers to a compound formed from a metal, oxygen and optionally other elements. Suitable metal oxides include, e.g., Copper(I) oxide (Cu₂O), Copper(II) oxide (CuO), Titanium(II) oxide (TiO), Zinc oxide (ZnO), Cobalt(II) oxide (CoO), Titanium dioxide (TiO₂), Titanium(III) oxide (Ti₂O₃), Manganese(VII) oxide (Mn₂O₇), Manganese(IV) oxide (MnO₂), Iron(III) oxide (Fe₂O₃), Iron(II) oxide (FeO), Nickel(III) oxide (Ni₂O₃), Nickel(II) oxide (NiO), Vanadium(V) oxide (V₂O₅), Vanadium(IV) oxide (VO₂), Vanadium(III) oxide (V₂O₃), Vanadium(II) oxide (VO), Tin dioxide (SnO₂), Tin(II) oxide (SnO), Indium(III) oxide (In₂O₃), ceria, barium titanate, bismuth ferrite and Barium oxide (BaO).

As used herein, “cerium(IV) oxide”, “ceric oxide,” “ceria,” “cerium oxide” or “cerium dioxide” refers to CeO₂.

As used herein, “barium titanate” refers to an oxide of barium and titanium with the chemical formula BaTiO₃.

As used herein, “bismuth ferrite” refers to an oxide of bismuth and iron, with the formula BiFeO₃.

As used herein, “transition metal oxide” refers to a compound formed from a transition metal, oxygen and optionally other elements. Transition metals include, e.g., zinc (Zn).

As used herein, “alkoxide” refers to the functional group O-alkyl, wherein alkyl refers to a C₁-C₃₀ hydrocarbon containing normal, secondary or tertiary carbon atoms. Examples include, e.g., methyl, ethyl, iso-propyl, etc.

As used herein, “halide” refers to F, Cl, Br or I.

As used herein, “ether group” refers to group—an oxygen atom connected to two (substituted) alkyl or aryl groups—of general formula R—O—R, wherein each R is independently alkyl or aryl.

As used herein, an “ether end group” refers to an ether group present at a terminal portion of a compound.

As used herein, a “metal salt precursor” is any compound containing a metal, capable of converting to the metal oxide, e.g., by alcoholysis. Suitable metal salt precursors include, e.g., metal acetates, metal citrates, metal oxalates, metal acetylacetonates, and metal alkoxides. Suitable specific metal salt precursors include, e.g., titanium acetylacetonate, titanium isopropoxide, zinc acetate, zinc citrate, zinc methacrylate, zinc oxalate, manganese acetate, cobalt acetate, and manganese acetylacetonate.

As used herein, “nanoparticle” refers to is a microscopic particle with at least one dimension less than 100 nm n.

As used herein, “crystalline” or “morphous” refers to solids in which there is long-range atomic order of the positions of the atoms.

As used herein, “amorphous” refers to a solid in which there is no long-range order of the positions of the atoms.

As used herein, “disperse” refers to the act of introducing solid particles in a liquid, such that the particles separate uniformly throughout the liquid.

As used herein, “redisperse” refers to the act of reintroducing solid particles in a liquid, such that the particles separate uniformly throughout the liquid.

As used herein, “monodisperse” refers to a narrow size distribution, such that the root mean square deviation from the diameter is less than about 10%. Specific metal oxide nanoparticles of the presently described subject matter are monodisperse.

As used herein, “highly monodisperse” refers to a narrow size distribution, such that the root mean square deviation from the diameter is less than about 5%. Specific metal oxide nanoparticles of the presently described subject matter are highly monodisperse.

As used herein, “surfactant” or “surface active agent” refers to wetting agents that lower the surface tension of a liquid, allowing easier spreading, and lower the interfacial tension between two liquids. Surfactants are typically classified into four primary groups; anionic, cationic, non-ionic, and zwitterionic (dual charge). A nonionic surfactant has no charge groups in its head. The head of an ionic surfactant carries a net charge. If the charge is negative, the surfactant is more specifically called anionic; if the charge is positive, it is called cationic. If a surfactant contains a head with two oppositely charged groups, it is termed zwitterionic.

As used herein, “inert gas” refers to any gas that is not reactive under normal circumstances. Unlike the noble gases, an inert gas is not necessarily elemental and are often molecular gases. Like the noble gases, the tendency for non-reactivity is due to the valence, the outermost electron shell, being complete in all the inert gases.

As used herein, “starting materials” or “starting materials of a chemical reaction” refers to those substances (i.e., compounds) that undergo a chemical transformation, under the specified conditions (e.g., time and temperature) and with the specified reagents and/or catalysts described therein.

As used herein, “contacting” refers to the act of touching, making contact, or of immediate proximity.

As used herein, “drying” includes removing a substantial portion (e.g., more than about 90 wt. %, more than about 95 wt. % or more than about 99 wt. %) of organic solvent and/or water present therein.

As used herein, “heating” refers to the transfer of thermal energy via thermal radiation, heat conduction or convection, such that the temperature of the object that is heated increases over a specified period of time.

As used herein, “room temperature” refers to a temperature of about 18° C. (64° F.) to about 22° C. (72° F.).

As used herein, “agitating” refers to the process of putting a mixture into motion with a turbulent force. Suitable methods of agitating include, e.g., stirring, mixing, and shaking.

As used herein, “atmospheric air” refers to the gases surrounding the planet Earth and retained by the Earth's gravity. Roughly, it contains nitrogen (75%), oxygen (21.12%), argon (0.93%), carbon dioxide (0.04%), carbon monoxide (0.07%), and water vapor (2%).

As used herein, “cooling” refers to transfer of thermal energy via thermal radiation, heat conduction or convection, such that the temperature of the object that is cooled decreases over a specified period of time.

As used herein, “polar solvent” refers to solvents that exhibit polar forces on solutes, due to high dipole moment, wide separation of charges, or tight association; e.g., water, alcohols, and acids. The solvents typically have a measurable dipole. Such solvents will typically have a dielectric constant of at least about 15, at least about 20, or between about 20 and about 30.

As used herein, “non-polar solvent” refers to a solvent having no measurable dipole. Specifically, it refers to a solvent having a dielectric constant of less than about 15, less than about 10, or between about 6 and about 10.

As used herein, “alcohol” includes an organic chemical containing one or more hydroxyl (OH) groups. Alcohols may be liquids, semisolids or solids at room temperature. Common mono-hydroxyl alcohols include, e.g., ethanol, methanol and propanol. Common poly-hydroxyl alcohols include, e.g., propylene glycol and ethylene glycol.

As used herein, “centrifuging” or “centrifugation” includes the process of separating fractions of systems in a centrifuge. The most basic separation is to sediment a pellet at the bottom of the tube, leaving a supernatant at a given centrifugal force. In this case sedimentation is determined by size and density of the particles in the system amongst other factors. Density may be used as a basis for sedimentation in density gradient centrifugation, at very high g values molecules may be separated, i.e. ultra centrifugation. In continuous centrifugation the supernatant is removed continuously as it is formed. It includes separating molecules by size or density using centrifugal forces generated by a spinning rotor. G-forces of several hundred thousand times gravity are generated in ultracentrifugation. Centrifuging effectively separates the sediment or precipitate from the fluid.

As used herein, “redispersing” refers to the act of introducing solid particles in a liquid, such that the particles separate uniformly throughout the liquid.

As used herein, “protic solvent” refers to a solvent that contains a dissociable H⁺ ion. Typically, the solvent carries a hydrogen bond between an oxygen (as in a hydroxyl group) or a nitrogen (as in an amine group).

As used herein, “aprotic solvent” refers to a solvent that lacks a dissociable H⁺ ion.

### Methods of Manufacturing (Processing)

In the methods of manufacturing described herein, the steps may be carried out in any order without departing from the principles of the disclosed subject matter, except when a temporal or operational sequence is explicitly described. Recitation in a claim to the effect that first a step is performed, then several other steps are subsequently performed, shall be taken to mean that the first step is performed before any of the other steps, but the other steps may be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D may be carried out in any sequence between steps A and E, and that the sequence still falls within the literal scope of the claimed process.

Furthermore, specified steps may be carried out concurrently unless explicit claim language recites that they be carried out separately. For example, a claimed step of doing X and a claimed step of doing Y may be conducted simultaneously within a single operation, and the resulting process will fall within the literal scope of the claimed process.

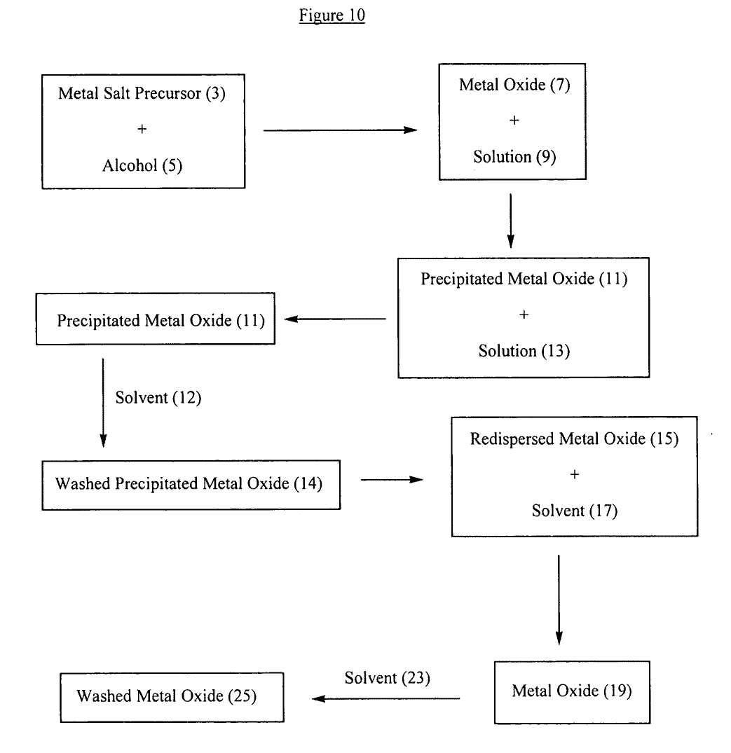

Referring to FIG. 10, methods to manufacture metal oxides of the disclosed subject matter are provided.

Briefly stated, FIG. 10 illustrates a method to manufacture a metal oxide (19) of the disclosed subject matter. The method includes contacting a metal salt precursor (3) and an alcohol (5), to provide a metal oxide (7) in solution (9). The metal oxide (7) is precipitated to provide precipitated metal oxide (11) in solution (13). The precipitated metal oxide (11) is removed from solution (13), and redispersed in solvent (17) to provide redispersed metal oxide (15). Optionally, upon removal from the solution (13), the precipitated metal oxide (11) is washed to provide the washed precipitated metal oxide (14), which is redispersed in solvent (17) to provide redispersed metal oxide (15). The redispersed metal oxide (15) is removed from the solvent (17) to provide metal oxide (19).

The metal salt precursor (3) and alcohol (5) may typically be contacted in any suitable manner, effective to provide the metal oxide (19). For example, the metal salt precursor (3) and alcohol (5) may be contacted while agitating. Additionally, the metal salt precursor (3) and alcohol (5) may be contacted for any suitable period of time, effective to provide the metal oxide (19). For example, the metal salt precursor (3) and alcohol (5) may be contacted for at least about 1 hour, at least about 5 hours, at least about 10 hours, at least about 24 hours or at least about 48 hours. Additionally, the metal salt precursor (3) and alcohol (5) may be contacted at any suitable temperature, effective to provide the metal oxide (19). For example, the metal salt precursor (3) and alcohol (5) may be contacted at a temperature of at least about 20° C., at least about 60° C., at least about 80° C. or at least about 100° C. Additionally, the metal salt precursor (3) and alcohol (5) may be contacted under one or more inert gases.

Both the metal salt precursor (3) and the alcohol (5) may be employed in any suitable amount and ratio, effective to provide the metal oxide (19). Specifically, the metal salt precursor (3) and alcohol (5) may be employed in a weight/volume (g/ml) ratio of about 1:100 to about 100:1, about 1:80 to about 80:1, about 1:50 to about 50:1, or about 1:20 to about 20:1, respectively. Alternatively, the alcohol (5) and metal salt precursor (3) may be employed in a volume/weight (ml/g) ratio of about 1:100 to about 100:1, about 1:80 to about 80:1, about 1:50 to about 50:1, or about 1:20 to about 20:1, respectively.

For example, the metal salt precursor (3) and alcohol (5) may be employed in a weight/volume (g/ml) ratio of about 0.0001 to about 1.0, about 0.001 to about 0.5 or about 0.001 to about 0.2.

Prior to contacting the metal salt precursor (3) and alcohol (5), the metal salt precursor (3) may be heated to a suitable temperature, and for a suitable period of time, effective to remove water. For example, the metal salt precursor (3) may be heated to a temperature of at least about 50° C., at least about 70° C., or at least about 90° C. Additionally, the metal salt precursor (3) may be heated for a period of time of at least about 10 minutes, at least about 20 minutes, at least about 30 minutes, or at least about 60 minutes. The dehydrated metal salt precursor (3) may include less than about 1 wt. % water, less than about 0.1 wt. % water, or less than about 0.001 wt. % water.

The metal oxide (7) may be precipitated in any suitable manner and under any suitable conditions, effective to provide precipitated metal oxide (11) in solution (13). The precipitation may occur at any suitable temperature, effective to provide precipitated metal oxide (11) in solution (13). For example, employing anhydrous ethanol (200 proof) as the alcohol (5), the precipitation may occur at a temperature of about 50° C. to about 120° C., about 70° C. to about 115° C., or about 90° C. to about 110° C.

Additionally, the precipitation may occur over any suitable period of time, effective to provide precipitated metal oxide (11) in solution (13). For example, the precipitation may occur over a period of time of at least about 1 hour, at least about 5 hours, at least about 10 hours, at least about 24 hours, or at least about 48 hours.

The precipitated metal oxide (11) may be removed from the solution (13) in any suitable manner. For example, the precipitated metal oxide (11) may be removed from the solution (13) by centrifuging and decanting the solution (13) from the precipitated metal oxide (11), by filtering the precipitated metal oxide (11) from the solution (13), or a combination thereof.

Upon separating the precipitated metal oxide (11) from the solution (13), the precipitated metal oxide (11) may optionally be washed with solvent (12), to provide a washed precipitated metal oxide (14). Any suitable solvent (12) may be employed, provided the solvent (12) removes a significant and appreciable amount of contaminants present with the precipitated metal oxide (11), and the solvent (12) does not dissolve a significant and appreciable amount of precipitated metal oxide (11). Suitable solvents (12) include, e.g., alcohols wherein suitable alcohols include, e.g., methanol, ethanol, propanol, butanol, pentanol, hexanol, heptanol, octanol, oleyl alcohol, sec-butanol, 2-ethyl hexyl alcohol, isobutanol, isopropanol, tert-butanol, cyclohexanol, 3-methoxy-1-butanol, 3-methoxy-1-propanol, methyl isobutyl carbinol, benzyl alcohol, and mixtures thereof.

The precipitated metal oxide (11) may be redispersed in any suitable solvent (17) and under any suitable conditions, effective to provide the redispersed metal oxide (15). For example, the precipitated metal oxide (11) may be redispersed by ultrasonification, effective to provide the redispersed metal oxide (15). The ultrasonification may be carried out for any suitable period of time, e.g., at least about 1 minute, at least about 10 minutes or at least about 30 minutes. Additionally, the solvent (17) may include at least one of water, a polar protic solvent, a polar aprotic solvent, a non-polar protic solvent, and a non-polar aprotic solvent. Specifically, the solvent (17) may include water or hexane.

The redispersed metal oxide (15) may be removed from the solvent (17) in any suitable manner, effective to provide the metal oxide (19). For example, the redispersed metal oxide (15) and solvent (17) may be centrifuged and the solvent (17) may be decanted. Alternatively, the redispersed metal oxide (15) may be filtered from the solvent (17).

Upon removing the redispersed metal oxide (15) from the solvent (17), the metal oxide (19) may optionally be washed with a suitable solvent (23), to provide washed metal oxide (25). The solvent (23) may include, e.g., a polar protic solvent, a polar aprotic solvent, a non-polar protic solvent, and a non-polar aprotic solvent, or a mixture thereof.

The disclosed subject matter may be illustrated by the following non-limiting examples.

### EXAMPLES

### Example 1

### Synthesis of Undoped Zinc Oxide Nanoparticles

To synthesize undoped ZnO nanoparticles, 0.3 gram of zinc acetate (purchased from Sigma-Aldrich) was mixed with 15 ml of 200 proof ethanol (purchased from Pharmco) at about 70° C. under stirring for 20 minutes to result in a clear solution. The clear solution was transferred to a Teflon-lined autoclave. The crystallization was carried out at a temperature of about 100° C. for about two to twelve hours under substantially static conditions. A cloudy suspension was observed and the resulting white product was collected by centrifugation followed by a thorough washing with ethanol.

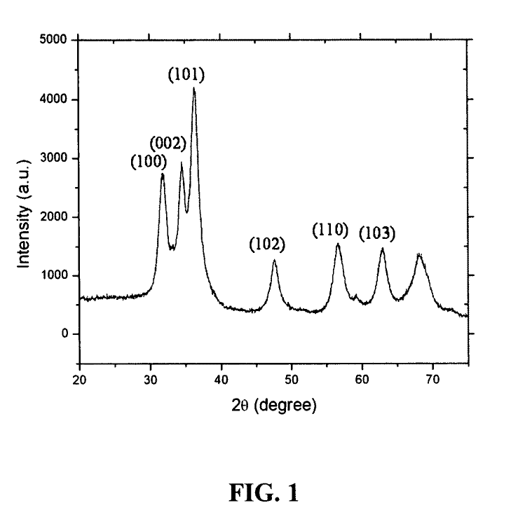

The precipitate was readily redispersible in water or hexane by ultrasonication to form a stable colloidal suspension. The as-collected wet white precipitates (with trace amount of ethanol) may be readily redispersed in water by ultrasonication for 1 minute to form a stable, quasi-transparent colloidal water suspension with concentration up to 10 wt %. No additional surfactants or additives were required. FIG. 1 shows an x-ray diffraction (XRD) pattern of the as-synthesized ZnO nanoparticles. XRD patterns were obtained with a Inel X-ray Diffractometer using Cu Kα radiation.

### Example 2

### Synthesis of Coated Zinc Oxide Nanoparticles

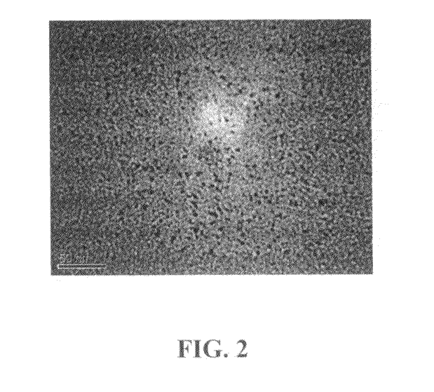

Oleic acid was coated on the surface of the ZnO nanoparticles by adding in drops of oleic acid into the wet precipitates of ZnO nanoparticles. This was followed by an ultrasonic treatment for about two minutes. Excess oleic acid was washed away with ethanol and the nanoparticles coated with oleic acid was re-dispersed in hexane to form a clear and stable solution. FIG. 2 shows a transmission electron microscope (TEM) image of the ZnO nanoparticles coated with oleic acid. TEM images were obtained using a high resolution transmission electron microscope (HRTEM) JEOL 3000F TEM/STEM.

### Example 2

### Synthesis of Doped Zinc Oxide Nanoparticles

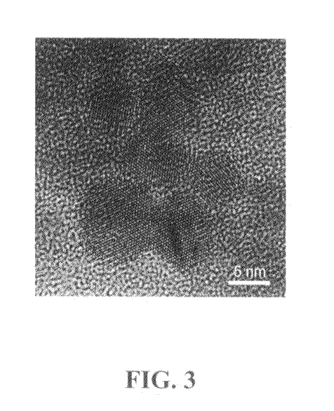

To synthesize Mn-doped ZnO nanoparticles, 1 gram of zinc acetate (purchased from Sigma-Aldrich) and 0.03 g of manganese acetate was mixed with 50 ml of 200 proof ethanol (purchased from Pharmco) at about 70° C. under stirring for 20 minutes to result in a clear solution. The clear solution was transferred to a Teflon-lined autoclave. The crystallization was carried out at a temperature of about 100° C. for about two to twelve hours under substantially static conditions. A cloudy suspension was observed and the resulting white product was collected by centrifugation followed by a thorough washing with ethanol. The precipitate was readily redispersible in water by ultrasonication to form a stable colloidal suspension. FIG. 3 shows a transmission electron microscope (TEM) image of the as-synthesized Mn-doped ZnO nanoparticles.

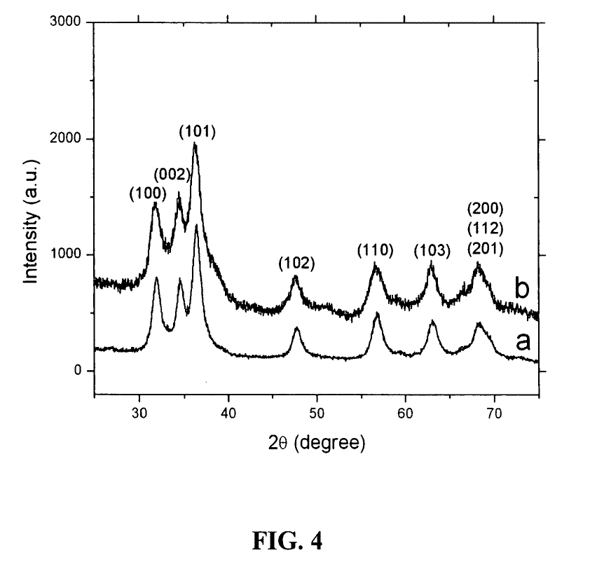

FIG. 4 shows the XRD spectra of ZnO nanoparticles (curve a) and Mn-doped (3 mol %) ZnO nanoparticles (curve b). As shown, the peaks match well with the Bragg reflections for standard wurtzite structure. The nanoscale size of the particles may be contributing to the broadness of the peaks, but both samples appear to show a high degree of crystallinity.

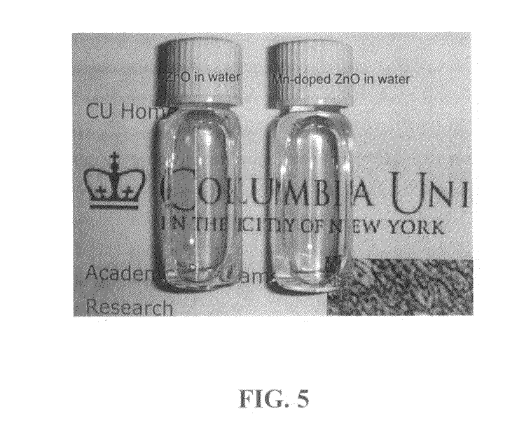

FIG. 5 show approximately 1 wt % ZnO nanoparticles dispersed in water and 1 wt % of Mn-doped (3 mol %) ZnO nanoparticles dispersed in water, without any additional surfactants or additives. As shown, the suspension is stable and transparent to the human eye. Stable and transparent concentrations up to (but not limited to) about 10 wt % is also possible.

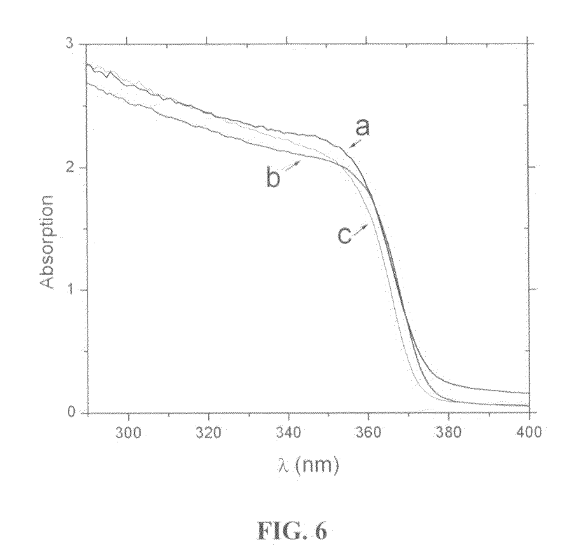

FIG. 6 shows a room temperature UV-vis absorption spectra of undoped ZnO crystallized for five hours (curve a), undoped ZnO crystallized for ten hours (curve b), and Mn-doped (3 mol %) ZnO nanoparticles crystallized for ten hours (curve c). Bulk ZnO typically has an absorption peak that is about 373 nm (3.32 eV) (not shown). ZnO and Mn-doped ZnO nanoparticles have absorption peaks around 355 to 360 nm. The pronounced blue shift in the absorption edges may be attributed to the quantum confinement effect arising from the nanoparticles. FIG. 6 further suggests UV-vis absorption characteristics of ZnO nanoparticles may be modified by chemical doping and crystal sizes variation using different crystallization temperatures and times. The UV-vis absorption spectra were collected on a HP 8453 UV/Visible Spectrophotometer.

Various other experiments were also conducted. For example, crystallization times were varied from about two to twelve hours. Differing amounts of manganese acetate (ranging from about 0.03 to 0.01 g) were utilized to form Mn-doped ZnO nanoparticles. Cobalt acetate was also utilized (instead of the manganese acetate) to form Co-doped ZnO nanoparticles.

In some other experiments, the crystallization of nanoparticles was carried out by transferring the clear solution described above to a well-sealed 250 ml plastic bottle in a water bath. The solution was then aged at about 60° C. for about 12 hours before heating up to about 80° C. until a cloudy suspension was observed. The whole mixture was then continually stirred at about the same temperature for about two additional hours. Without wishing to be bound by theory, the stirring process may improve the diffusion in solution and thus favor the formation of ZnO nanocrystals under relatively low crystallization temperature.

### Example 3

### Synthesis of Undoped TiO₂ Nanoparticles

To synthesize undoped TiO₂ nanoparticles, 0.3 gram of titanium (IV) oxide acetylacetonate (TiO(acac)₂) was mixed with 15 ml of 200 proof ethanol (purchased from Pharmco) at about 70° C. under stirring for about 20 minutes to result in a yellowish suspension. The suspension was transferred to a Teflon-lined autoclave. The crystallization was carried out at a temperature of about 180° C. for about 24 hours under substantially static conditions. A cloudy suspension was observed and the resulting white or light yellowish product was collected by centrifugation followed by a thorough washing with ethanol. The precipitate was readily redispersible in water by ultrasonication to form a stable colloidal suspension.

Similarly, 0.3 gram of titanium (IV) oxide acetylacetonate (TiO(acac)₂) was mixed with 15 ml of oleyl alcohol (purchased from Aldrich) at about 70° C. under stirring for about 20 minutes to result in a yellowish suspension. The suspension was transferred to a Teflon-lined autoclave. The crystallization was carried out at a temperature of about 180° C. for about 24 hours under substantially static conditions. A cloudy suspension was observed and the resulting white or light yellowish product was collected by centrifugation followed by a thorough washing with ethanol. The precipitate was readily redispersible in hexane by shaking to form a clear and stable solution.

Alternatively, 0.3 gram of titanium isopropoxide may be mixed with 15 ml of 200 proof ethanol at about 70° C. under stirring for about 20 minutes to result in a clear solution. The clear solution may be transferred to a Teflon-lined autoclave. The crystallization may be carried out at a temperature of about 180° C. for about 24 hours under substantially static conditions. When a cloudy suspension is observed, and the resulting white or light yellowish product may be collected by centrifugation followed by a thorough washing with ethanol. The precipitate may be readily redispersible in water by ultrasonication to form a stable colloidal suspension.

### Example 3

### Synthesis of Doped TiO₂ Nanoparticles

Various other experiments were also conducted. For example, to form Mn-doped TiO₂ nanoparticles, 0.3 g of TiO(acac)₂ and 0.003 to 0.009 g of Mn(acac)₂ was mixed with 15 ml of ethanol at about 70° C. under stirring conditions. The doping levels may vary in the range of 1 to 3 mol %. TiO₂ and doped TiO₂ nanoparticles with varied sizes were also synthesized using a mixture of ethanol and other alcohol such as oleyl alcohol. Cobalt acetate was also utilized (instead of the manganese acetate) to form Co-doped ZnO nanoparticles.

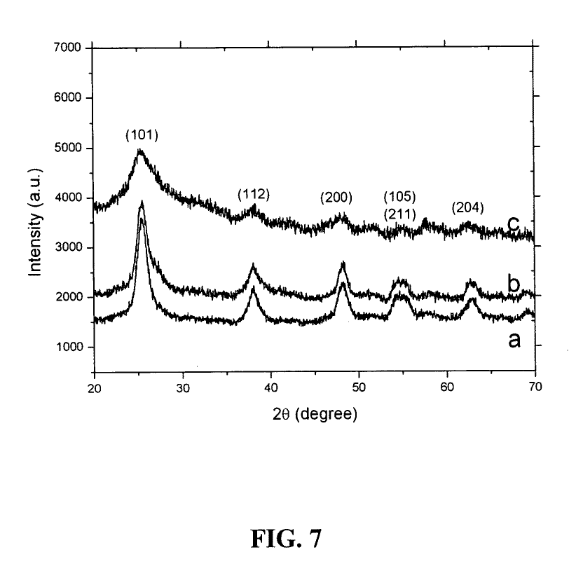

FIG. 7 shows an x-ray diffraction (XRD) pattern of the TiO₂ nanoparticles synthesized in ethanol, Mn-doped (3 mol %) TiO₂ nanoparticles synthesized in ethanol, and TiO₂ nanoparticles synthesized in oleyl alcohol. The broader peaks of the TiO₂ nanoparticles synthesized in oleyl alcohol may be attributed to the smaller diameter of the TiO₂ nanoparticles that form. Without wishing to be bound by theory, utilizing alcohols with a longer backbone, such as oleyl alcohol over ethanol, may produce smaller nanoparticles because the long chain alcohol may absorb on the particle surface to stabilize the nanoparticles.

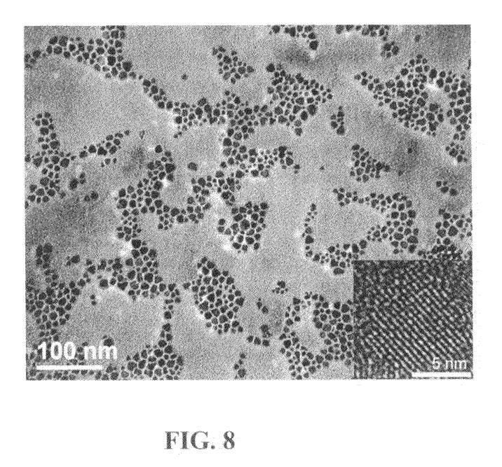

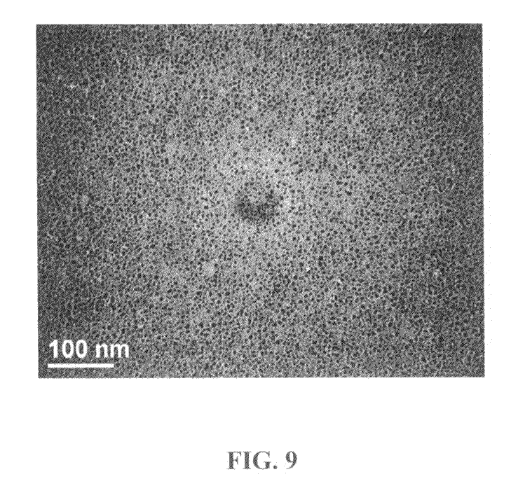

FIG. 8 and FIG. 9 show TEM images of the TiO₂ nanoparticles synthesized in ethanol and in oleyl alcohol, respectively. The TEM results further confirm that the nanoparticles synthesized using oleyl alcohol has on average a smaller diameter.

In some other experiments, the crystallization of nanoparticles was carried out by transferring the clear solution described above to a well-sealed 250 ml plastic bottle in a water bath. The solution was then aged at about 60° C. for about 12 hours before heating up to about 80-100° C. until a cloudy suspension was observed. The whole mixture was then continually stirred at about the same temperature for about two additional hours.

All publications, patents, and patent applications cited herein are incorporated herein by reference in their entirety. While in the foregoing specification this invention has been described in relation to certain preferred embodiments thereof, and many details have been set forth for purposes of illustration, it will be apparent to those skilled in the art that the invention is susceptible to additional embodiments, combinations and sub-combinations; and that certain of the details described herein may be varied considerably without departing from the basic principles of the invention.

1. A method for preparing a metal oxide, the method comprising:
(a) contacting a metal salt precursor with an alcohol to provide a metal oxide; and
(b) removing the metal oxide from the alcohol.

2. The method of claim 1, wherein the metal salt precursor comprises at least one of a metal acetate, metal citrate, metal oxalate, metal acetylacetonate, and a metal alkoxide.

3. The method of claim 1, wherein the metal salt precursor comprises at least one of titanium acetylacetonate, titanium isopropoxide, zinc acetate, zinc citrate, zinc methacrylate, zinc oxalate, manganese acetate, cobalt acetate, and manganese acetylacetonate.

4. The method of claim 1, wherein the alcohol comprises at least one of methanol, ethanol, propanol, butanol, pentanol, hexanol, heptanol, octanol, oleyl alcohol, sec-butanol, 2-ethyl hexyl alcohol, isobutanol, isopropanol, tert-butanol, cyclohexanol, 3-methoxy-1-butanol, 3-methoxy-1-propanol, methyl isobutyl carbinol, and benzyl alcohol.

5. The method of claim 1, wherein the metal salt precursor and the alcohol are contacted for a period of time of at least about 10 hours.

6. The method of claim 1, wherein the metal salt precursor and the alcohol are contacted at a temperature of at least about 60° C.

7. The method of claim 1, wherein the metal salt precursor and the alcohol are contacted while agitating.

8. The method of claim 1, wherein the contacting the metal salt precursor with the alcohol provides a metal oxide that precipitates from the alcohol.

9. The method of claim 1, wherein the contacting the metal salt precursor with the alcohol provides a metal oxide that crystallizes from the alcohol.

10. The method of claim 1, wherein the removing the metal oxide from the alcohol comprises centrifuging the metal oxide and the alcohol, decanting the alcohol, and optionally washing the metal oxide with additional alcohol.

11. The method of claim 1, wherein the removing the metal oxide from the alcohol comprises centrifuging the metal oxide and the alcohol, filtering the metal oxide, and optionally washing the metal oxide with additional alcohol.

12. The method of claim 1, further comprising after the removing the metal oxide from the alcohol, redispersing the metal oxide in a solvent to provide a colloidal suspension of the metal oxide and the solvent.

13. The method of claim 12, further comprising separating the metal oxide and the solvent.

14. The method of claim 13, wherein the solvent comprises at least one of water, a polar protic solvent, a polar aprotic solvent, a non-polar protic solvent, and a non-polar aprotic solvent.

15. The method of claim 1, wherein the metal salt precursor does not include alkoxide or halide ligands.

16. The method of claim 1, wherein the metal oxide comprises at least one transition metal oxide.

17. The method of claim 1, wherein the metal oxide comprises at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.

18. The method of claim 1, further comprising after the removing the metal oxide from the alcohol, contacting the metal oxide and a pharmaceutical carrier or diluent.

19. The method of claim 1, further comprising after the removing the metal oxide from the alcohol, contacting the metal oxide and a cosmetic carrier or diluent.

20. The method of claim 1, wherein the metal oxide obtained is a nanoparticle.

21. The method of claim 1, wherein the metal oxide obtained has a functionalized surface.

22. The method of claim 1, wherein the metal oxide obtained is terminated with one or more ether end groups.

23. The method of claim 1, wherein the metal oxide obtained is modified or coated with one or more capping agents.

24. The method of claim 1, wherein the metal oxide obtained is about 0.1 nm to about 100 nm in diameter.

25. The method of claim 1, wherein the metal oxide obtained is about 0.1 nm to about 50 nm in diameter.

26. The method of claim 1, wherein the metal oxide obtained is about 5 nm to about 20 nm in diameter.

27. The method of claim 1, wherein at least two metal salt precursors are employed, such that the metal oxide that is obtained is doped with at least one additional metal.

28. A method for preparing a metal oxide nanoparticle, the method comprising:
(a) contacting a metal salt precursor with an alcohol to provide a metal oxide;
(b) removing the metal oxide from the alcohol;
(c) redispersing the metal oxide in a solvent to provide a colloidal suspension of the metal oxide and the solvent; and
(d) removing the metal oxide from the solvent to provide a metal oxide nanoparticle comprising at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.

29. A method for preparing a metal oxide nanoparticle, the method comprising:
(a) contacting two or more metal salt precursors with an alcohol to provide a metal oxide that precipitates from the alcohol, wherein the metal salt precursor comprises at least one of titanium acetylacetonate, titanium isopropoxide, zinc acetate, zinc citrate, zinc methacrylate, zinc oxalate, manganese acetate, cobalt acetate, and manganese acetylacetonate;
(b) removing the precipitated metal oxide from the alcohol;
(c) redispersing the precipitated metal oxide in a solvent to provide a colloidal suspension of the redispersed metal oxide and the solvent; and
(d) removing the redispersed metal oxide from the solvent to provide a metal oxide nanoparticle comprising at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.

30. A method for preparing a metal oxide nanoparticle, the method comprising:
(a) contacting a metal salt precursor with an alcohol to provide a metal oxide that precipitates from the alcohol, wherein the metal salt precursor comprises at least one of titanium acetylacetonate, titanium isopropoxide, zinc acetate, zinc citrate, zinc methacrylate, zinc oxalate, manganese acetate, cobalt acetate, and manganese acetylacetonate;
(b) removing the precipitated metal oxide from the alcohol;
(c) redispersing the precipitated metal oxide in a solvent to provide a colloidal suspension of the redispersed metal oxide and the solvent; and
(d) removing the redispersed metal oxide from the solvent to provide a metal oxide nanoparticle comprising at least one of titanium oxide, zinc oxide, copper oxide, cobalt oxide, manganese oxide, iron oxide, nickel oxide, vanadium oxide, tin oxide, indium oxide, ceria, barium titanate, and bismuth ferrite.
